(19) United States
(12) Patent Application Publication
(10) Pub. No.: US 2014/0377388 A1
(43) Pub. Date: Dec. 25, 2014

(54) CHOLESTEROL DISCHARGE-ACCELERATING AGENT AND NEUTRAL FAT DISCHARGE-ACCELERATING AGENT USING SUNFLOWER PITH

(71) Applicants: Yoshio Takamura, Tokyo (JP); Masaru Takumi, Tochigi (JP); Nakamichi Watanabe, Tokyo (JP); Mana Fujimoto, Tokyo (JP); Ako Okano, Tokyo (JP); Junko Sakurai, Tokyo (JP); Yoshihiro Okada, Hiroshima (JP); Toshiharu Nagai, Tokyo (JP)

(72) Inventors: Yoshio Takamura, Tokyo (JP); Masaru Takumi, Tochigi (JP); Nakamichi Watanabe, Tokyo (JP); Mana Fujimoto, Tokyo (JP); Ako Okano, Tokyo (JP); Junko Sakurai, Tokyo (JP); Yoshihiro Okada, Hiroshima (JP); Toshiharu Nagai, Tokyo (JP)

(73) Assignee: MOTTAINAI BIOMASS RESEARCH CORPORATION, Chuo-ku, Tokyo (JP)

(21) Appl. No.: 14/368,956

(22) PCT Filed: Sep. 11, 2012
(86) PCT No.: PCT/JP2012/073173

§ 371 Date: Jun. 26, 2014

(30) Foreign Application Priority Data
Dec. 28, 2011 (JP) 2011-287233

(51) Int. Cl. A61K 36/28; A23L 1/308

(52) U.S. Cl. CPC A61K 36/28; A23L 1/3081
USPC 424/764

(57) ABSTRACT

An object of the present invention is to provide a safe and inexpensive substance exhibiting the effects of discharging in vivo cholesterol and neutral fat. The present inventors have discovered that the above object can be achieved using the sunflower stem internal fibers (pith) of plants of the family Compositae. According to the present invention, the discharge of in vivo neutral fats and cholesterol can be accelerated and thus excellent slimming effects can also be obtained.

### TECHNICAL FIELD

The present invention provides substances exhibiting beneficial effects on the discharge of in vivo cholesterol and neutral fat using sunflower stem internal fibers.

### BACKGROUND ART

Dietary fibers are recognized as having intestinal regulatory effects, and thus the intake of large amounts of dietary fibers is recommended. Since dietary fibers have no taste and are dry, the daily intake of 5 g to 10 g of dietary fibers is extremely difficult. As a result, many people (and women in particular) suffer from constipation because of dietary fiber deficiencies. Medication or enemas are mainly employed for treatment of constipation, but are problematic in terms of stomachache, habituation, and the like.

A main supply source of dietary fibers is crystallized cellulose from wood pulp. Examples thereof include vegetable foods such as cereals, beans, fruits, and seeds. Specific examples thereof include wheat, rice, and corn. Barley, rye, millet, sorghum and the like are also included herein.

Meanwhile, sunflower seeds are mainly cultivated as raw materials for vegetable oil in European regions around Russia. Of the cases reported in Oil World magazine, 29.84 million tons of sunflower seeds were produced, and 11.171 million tons of sunflower oil was produced during 2006 to 2007.

Sunflower oil is marketed in Japan, although on a small scale, such as in Hokuryu-cho (Hokkaido), Osaki-shi (Miyagi-ken), Namegata-shi (Ibaraki-ken), Nanko-cho (Hyogo-ken) and Hikawa-cho (Tottori-ken). Sunflower is cultivated throughout almost all of Japan, if sunflowers used for ornamental purposes are included, and it is a crop that can be easily cultivated. Test production runs of sunflower as a raw material for a biodiesel fuel have been conducted in some areas. However, the yield of sunflower seeds is as low as 200 kg/10 ares, and agricultural profits therefrom cannot be expected. Thus, sunflower is not widely cultivated.

However, most sunflower stems are mixed with soil in the field as compost. Sunflower stems are used as oil adsorption material in only a few cases in Southeast Asia. The dry weight of the sunflower stem portion is about ⅓ the seed portion. Thus sunflower stems are portions that poorly attract attention as a biomass resource.

Patent documents relating to sunflower are as examined as follows. JP Patent Publication (Kokai) No. 2005-60366 A (patent document 1) describes “an agent for accelerating lipolysis, comprising as an active ingredient a plant selected from common juniper, . . . sunflower . . . knotgrass (Polygonum aviculare), or an extract thereof.” Also, JP Patent Publication (Kokai) No. 2009-242432 A (patent document 2) describes “an agent for accelerating lipolysis, comprising as an active ingredient a plant selected from togenashi (Rosa roxburghii), . . . sunflower, . . . knotgrass (Polygonum aviculare), or an extract thereof.” Both documents describe that in the case of sunflower, seeds are preferably used.

Moreover, JP Patent Publication (Kohyo) No. 2002-504359 A (patent document 3) describes a method for producing a fat mixture for decreasing cholesterol, wherein sunflower oil is used as a type of oil. Furthermore, JP Patent Publication (Kokai) No. 2006-22068 A (patent document 4) describes the invention of a nutrient composition for improvement of serum lipid metabolism, wherein high oleic sunflower oil is used.

JP Patent Publication (Kohyo) No. 2006-517222 A (patent document 5) describes oil enriched with diacylglycerol and phytosterol ester, such as sunflower oil used for decreasing cholesterol and triglyceride.

### CITATION LIST

### Patent Documents

Patent Document 1: JP Patent Publication (Kokai) No. 2005-060366 A
Patent document 2: JP Patent Publication (Kokai) No. 2009-242432 A
Patent document 3: JP Patent Publication (Kohyo) No. 2002-504359 A
Patent document 4: JP Patent Publication (Kokai) No. 2006-022068 A
Patent document 5: JP Patent Publication (Kohyo) No. 2006-517222 A
Patent document 6: JP Patent Publication (Kokai) No. 2010-116344 A

### SUMMARY OF THE INVENTION

### Problem to be Solved by the Invention

An object of the present invention is to develop a safe and inexpensive substance for accelerating cholesterol discharge and a safe and inexpensive substance for accelerating neutral fat discharge, which exhibit excellent slimming effects by accelerating the discharge of in vivo cholesterol and neutral fat to the outside the body.

### Means for Solving the Problem

As a result of intensive studies to achieve the above object, the present inventors have discovered that the object can be achieved by separating a sunflower stem into epidermal rinds and stem internal fibers (pith) as shown in FIG. 5, with the use of a separator, drying the solid portion using a vacuum drying apparatus or a general drying system (e.g., solar drying or hot air drying), and then grinding the solid portion and using the resultant.

Specifically, the present invention relates to:

(1) a cholesterol and/or neutral fat discharge-accelerating agent, comprising stem internal fibers (pith) separated from a sunflower stem as a major ingredient;
(2) a food containing stem internal fibers (pith) separated from a sunflower stem as a major ingredient;
(3) a supplement containing stem internal fibers (pith) separated from a sunflower stem as a major ingredient; and
(4) a method for producing a cholesterol and/or neutral fat discharge-accelerating agent, comprising the steps of separating a sunflower stem into an stem external portion (rind) and stem internal fibers (pith), and then drying and grinding the stem internal fibers (pith).

The term “stem internal fibers (pith)” as used herein refers to microporous fibers that are passage ways for the moisture and nutrients of the core portion separated from rinds using a cane separator. When a plant stem is separated into the surface, rinds, sclerenchyma, vascular bundles, and pith, the term “stem internal fibers (pith)” is a generic name of a product prepared by grinding the vascular bundle and pith.

The present inventors have discovered that lipids are also present in the sunflower stem portions through a test on paper pulp. In particular, they have focused on the fact that fats and oils that are finally stored in seeds or raw materials thereof are transported to an stem internal portion (pith) via the interior of the stem (pith). They have determined that the lipophilic functions of the stem internal portion of sunflower can be expected to exhibit slimming effects, as a raw material for food. Thus, they have conducted experiments. As part of such process, they have previously applied for a patent (JP Patent Publication (Kokai) No. 2010-116344 A (patent document 6)) concerning oral agents and foods having functions of removing enteral waste products containing plant fruit fibers.

Use of the stem portions of many fast growing plants on earth is problematic in terms of the high cost required for drying to remove moisture accounting for nearly 70%-90% of the stem portions and the resulting high cost for grinding. Sugarcane or sweet sorghum (Sorghum bicolor) with a high sugar content is merely industrially cultivated to obtain stems. However, the present inventors have discovered that even sunflower, from which no sugar can be expected, can be used as a food having slimming effects by separating a sunflower plant into the rind portion and the stem internal portion (pith) using a cane separator, drying the stem internal portion using a vacuum drying apparatus or a general drying system (e.g., solar drying or hot air drying) to result in a moisture content suitable for grinding (preferably 8% or less), and then using the lipophilicity of the thus dried stem internal portion (pith).

Furthermore, sunflower is an agricultural product that can be cultivated without the use of any agricultural chemicals. The pith portions of sunflower stems consist only of water separated from the plants. With the use of this advantage, the plant water of sunflower stems (which conventionally have been discarded by transpiration) is separated using a distilling apparatus (vacuum drying unit) in a drying step. The thus produced product can be used as a valuable liquid product with high permeability, such as cosmetics, beverage water, and the like. In this manner, the possibility of using stems comprising pith, rind, and sunflower water has been discovered.

Stem internal fibers can be extracted from sunflower by: separating rind portions of the epidermis from pith (interior tissue) using a separation apparatus such as a cane separation system (AmClyde, U.S.A. U.S. Pat. No. 3,690,358) as shown in FIG. 6, performing solid-liquid separation using a vacuum drying unit (F·E·C); drying the solid portion to facilitate grinding to result in a moisture content of 15% or less and preferably 8% or less; and then grinding the dry solid portion using a grinder such as an impact grinder.

When the thus obtained stem internal fibers are used as foods, in addition to the effects of accelerating cholesterol or neutral fat discharge, various effects such as oil absorption power, water absorption power, and binding effects can be obtained. For example, stem internal fibers mixed into bread provide food with a soft texture in the mouth while suppressing elongation, and such fibers mixed into cookies provide wet texture. Moreover, stem internal fibers can also be added to supplements, supplement excipients, cereals, bolo, ice cream, and the like.

According to the present invention, dietary fibers obtained from sunflower stem internal fibers can be included in a food so as to account for about 20% of such food, and such fibers can be included in the form of an excipient for a supplement so as to account for about 90% of the agent. For taste-related reasons, conventional dietary fibers only account for up to 3% of food content. Unlike such conventional dietary fibers, the dietary fibers of the present invention can be included in significantly large amounts in food. Thus, the dietary fibers of the present invention are able to exhibit significant effects on improvement of cholesterol and/or neutral fat discharge, for example, and are able to enhance slimming effects.

Some or all of the content disclosed in the description and/or drawings of Japanese Patent Application No. 2011-287233, which is a priority document of the present application, is herein incorporated by reference.

### Effects OF THE INVENTION

The present invention is able to exhibit significant effects on improvement of cholesterol and/or neutral fat discharge and is able to enhance slimming effects.

### BRIEF DESCRIPTION OF THE DRAWINGS

FIG. 1 shows the results of body weight loss due to sunflower stem internal fibers.

FIG. 2 Shows decreases in white adipose tissue due to sunflower stem internal fibers.

FIG. 3 shows a decrease in triglyceride due to sunflower stem internal fibers.

FIG. 4 shows decreases in cholesterol due to sunflower stem internal fibers.

FIG. 5 shows a cross section of sunflower.

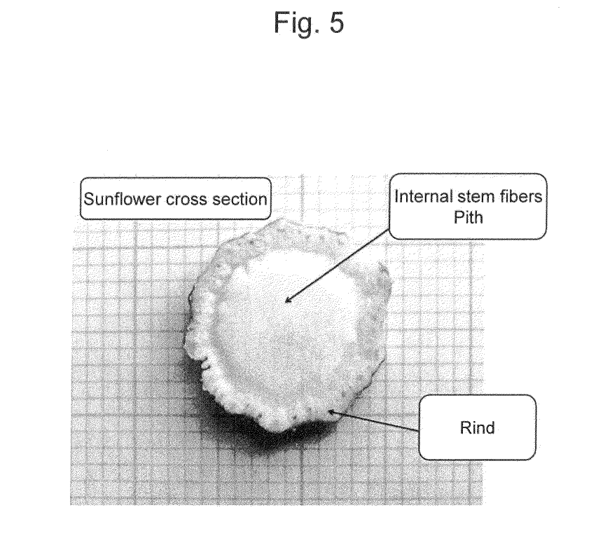

FIG. 6 shows a cane separator.

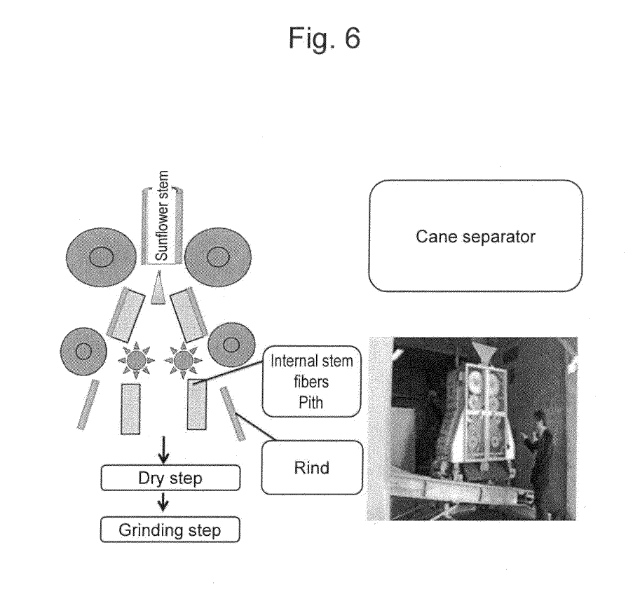

### EMBODIMENTS FOR CARRYING OUT THE INVENTION

Hereinafter, the present invention is more specifically described using Examples, but the scope of the present invention is not limited to these Examples.

### Example 1

### Method for Producing Sunflower Stem Internal Fiber

1. Sunflower Stems were Fed Intact into a Cane Separator (Mitsubishi Paper Mills Limited).
2. Sunflower stems fed into the separation apparatus were cracked in half and then the stem internal portion (pith) was excised with a rotary blade.
3. A stem internal portion (pith) with a moisture content ranging from 50% to 90% was subjected to solid-liquid separation using a vacuum drying system with a vacuum between −95 kPa and −98 kPa (F·E·C). As a result, a solid portion with a moisture content of 8% or less, which was most suitable for grinding, could be separated from liquid plant water.
4. It became possible to easily and finely grind the separated stem internal portion to result in a water content of 8% or less and a size of 125 microns or less with a general hammer mill, cyclone mill, or the like, regardless of grinder types. Thus it became possible to obtain ground particles that could be mixed with bread or the like.
5. Separated plant water is moisture extracted via a plant bio filter, which is applicable to beverages and cosmetics with small clusters and good absorbency.

### Example 2

Sunflower stem internal fiber powder was added (5% or 10%) to wheat flour, and thus miniature pieces of bread was baked.

The composition of the bread is as follows.

1) With respect to domestic hard flour (100), sunflower pith fibers (5), table sugar (2), salt (1.2), salt-free butter (4), natural yeast (6), and water (67)
2) With respect to domestic hard flour (100), sunflower pith fibers (10), table sugar (2), salt (1.2), salt-free butter (4), natural yeast (6), and water (67)

The resulting bread described above was light brown and it was found to have a soft texture in the mouth when tasted in all cases, compared with general types of bread. Bread baked with the okara (bean curd refuse) or bread with bran content of at least 3% had a dry, paper-like texture in the mouth. However, bread containing sunflower stem internal fiber powder had good affinity for other materials. Thus, bread having soft texture in the mouth could be produced, even if the sunflower stem internal fiber powder was added to account for 10% with respect to the wheat flour.

Therefore, through addition of sunflower pith fibers, the texture in the mouth was improved, cholesterol discharge and neutral fat discharge could be accelerated, and thus slimming effects could be enhanced.

### Example 3

### Cookies

Two types of cookies containing sunflower stem internal fiber powder were produced by varying the content of sunflower stem internal fiber powder, and then they were compared.

With respect to soft flour (100),
table sugar (30)
salt-free butter (50), and
whole egg (7.5)
were mixed to prepare a base for cookies. Sunflower stem internal fiber powder was mixed to account for 5% or 10% of the soft flour, so that 2 types of cookies were prepared.

Two types of cookies with the above composition were evaluated by tasting. As a result, both types of cookies were confirmed to have a wet (not dry) texture similar to that of general fiber-free cookies. Cookies with the general crystallized cellulose, bran, or okara (bean curd refuse) content of 3% were dry and thus tasted unpleasant. However, it was revealed that high-concentration fiber sweets having good affinity for other materials and slimming effects can also be produced.

### Example 4

### Supplement

### (Pill)

| | | |
|---|---|---|
| Sunflower pith stem internal fiber powder | 40 | mg |
| Sugarcane pith | 38 | mg |
| Uncentrifuged sugar | 106 | mg |
| Kanbaiko (rice powder) | 16 | mg |

With the above composition, 200 mg of pills were prototyped. As a result, water could be easily added to the pills and the pills had strong binding effects, so that they could be easily kneaded into a round shape.

### (Granule)

| | |
|---|---|
| Sunflower pith stem internal fiber powder | 50% |
| Uncentrifuged sugar | 50% |
| Mesh size | 30 mesh, screen 0.8 mm |

Uncentrifuged sugar (brand: Kinarito) was used as a binder in fluffy sunflower pith, enabling a sunflower pith stem internal fiber powder content of 50% while maintaining a pleasant taste.

### Example 5

### Composition Ratio of Sunflower Sterols

### (Method)

Sunflower (stem external portion), sunflower (stem internal fiber portion), and sunflower (seed portion) were each (50 g each) crushed with a food processor, a chloroform-methanol (2:1, v/v) mixture (300 mL) was added to the crushed product, and then the resultant was stirred with a magnetic stirrer for extraction. An extract was separated by filtration. Similar extraction procedures were repeated twice using the residue. The extract was collected, the solvent was evaporated under reduced pressure, the weight was measured, and the result was designated as the total lipid weight. Furthermore, a portion thereof was subjected to gas chromatography analysis, so as to determine the lipid class composition. The results are summarized in Table 1.

TABLE 1

| | Sterols | | | |
|---|---|---|---|---|
| Sample name | Campesterol | Stigmasterol | Sitosterol | Total |
| Sunflower | 2.8 | 2.2 | 2.5 | 7.5 |
| (Stem external | | | | |
| portion (rind)) | | | | |
| Sunflower | 3.6 | 13.3 | 4.4 | 21.3 |
| (Stem internal fiber | | | | |
| portion (pith)) | | | | |
| Sunflower | 0 | 0 | 0.4 | 0.4 |
| (Seed portion) | | | | |

As understood from Table 1, the sunflower “stem internal fiber portion (pith)” had a higher “stigmasterol” content than the “sunflower stem external portion,” and the sunflower “seed portion” contained almost no stereol as an ingredient.

Specifically, all of the inventions described in prior patent documents 1, 2, 3, 4, and 5 comprise the use of sunflower seeds, so that, regarding sterols, sitosterol is contained only in low amounts. The composition ratios of sterols in these prior patent documents completely differ from those of the present invention comprising the use of the sunflower “stem internal fiber portion (pith).”

The plant sterols campesterol, stigmasterol, and sitosterol have the property of not easily entering animal blood. Moreover, plant sterols are known to have the effect of adsorbing and excreting cholesterol since they have molecular structures analogous to animal sterol (cholesterol). Therefore, sunflower stem internal fibers are able to exhibit the effect of adsorbing cholesterol and causing cholesterol discharge to a greater degree than the sunflower stem external portion or seeds. Moreover, it was further proven that regarding the carrier functions of sterols to transport nutrients, seeds do not require such functions and thus contain no sterol, while stems must transport nutrients and thus are rich in sterols.

### Example 6

### Test for Comparison of Sugarcane Pith, Kao-Liang, and Sunflower Stem Internal Fiber Powder for Oil Absorption

### (Method)

1.0 g of sample powder and 20 mL of high oleic safflower oil were added to a 50-mL centrifugation tube. After 1 minute of shaking and mixing, centrifugation was performed at 1000 g for 3 minutes. Separated oil was collected and then the volume was measured.

### (Result)

### Comparison of Oil Absorption

| | | Sunflower | | |
|---|---|---|---|---|
| | Sugarcane | stem internal | Kao- | VITACEL |
| | pith | fiber powder | liang | WF600 |
| Mixed oil; | 20 | 20 | 20 | 20 |
| mL | | | | |
| Collected | 14 | 7 | 15 | 16 |
| oil; mL | | | | |
| Adsorbed | 6 | 13 | 5 | 4 |
| oil; mL | | | | |

As a result of a comparative study for oil absorption using the above method, the “sunflower stem internal fiber powder” was found to have adsorbed high oleic safflower oil to an extent 2- to 3-fold higher than sugarcane pith, kao-liang, or VITACEL. Thus, it can be said that the sunflower stem internal fiber powder had very high lipophilicity. In addition, VITACEL was crystallized cellulose (produced in Germany) comprising 99% or more cellulose, with is the same level as that of cellulose powder used as a control in Example 7 or later.

### Example 7

A mouse high-fat feed (lipid 20%), AIN93G (standard purified feedstuff composition for nutritional study for mice and rats, published by the American National Institute of Nutrition 1993), was mixed with the sunflower stem internal fiber (pith) powder (125 microns or less) so that the content of the dietary fiber portion (67.4 g/100 g) would be 5% or 10%. Cellulose powder (crystallized cellulose, Oriental Yeast Co., Ltd.) generally used for testing mice was mixed for comparison so that the content would be similarly 5% or 10%. This feed was continuously administered to 4 types of mice (4 groups×6 mice/group) for 3 weeks, and then differences in body weight were measured.

| AIN-93G | Composition (%) |
|---|---|
| Casein | 20.0 |
| L-cystine | 0.3 |
| Corn starch | 39.7486 |
| | (34.7486) |
| α-corn starch | 13.2 |
| Sucrose | 10.0 |
| Soybean oil | 7.0 |
| Cellulose powder | 5.0 |
| | (10.0) |
| AIN-mineral mixed | 3.5 |
| AIN-vitamin mixed | 1.0 |
| Choline bitartrate | 0.25 |
| Tertiary butylhydroquinone | 0.0014 |

Within the above composition, figures within parentheses were employed when the concentration of cellulose powder in a feed was as high as 10%.

Cellulose powder was compared with the sunflower stem internal fibers (pith) for composition. Whereas cellulose powder was composed of 99% or more cellulose, the sunflower stem internal fibers (pith) were composed as follows (analyzed by Japan Food Research Laboratories).

| | | |
|---|---|---|
| Cellulose | 35 | g/100 g |
| Hemicellulose | 9 | g/100 g |
| Lignin | 1.8 | g/100 g |
| Dietary fibers | 67.4 | g/100 g |
| Ash content | 12.4 | g/100 g |
| Lipids | 0.8 | g/100 g |

Based on the above information, it can be understood that the composition is characterized in particular by the fact that it contains hydrophilic hemicellulose and lipophilic lipids (e.g., plant sterols).

Mice used in this example were healthy C57-B2/6J mice. AIN93G, a high-fat feed (standard purified feedstuff composition for nutritional study for mice and rats, published by the American National Institute of Nutrition 1993) with a 20%-lipid composition, was used.

1) Control 1 group: 6 mice, with 5% crystallized cellulose powder incorporated into the feed.
2) Sunflower 1 group: 6 mice, with the 5% dietary fiber portion (67.4 g/100 g) of the sunflower stem internal fiber powder incorporated into the feed.
3) Control 2 group: 6 mice, with 10% crystallized cellulose powder incorporated into the feed.
4) Sunflower 2 group: 6 mice, with the 10% dietary fiber portion (67.4 g/100 g) of sunflower stem internal fiber powder incorporated into the feed.

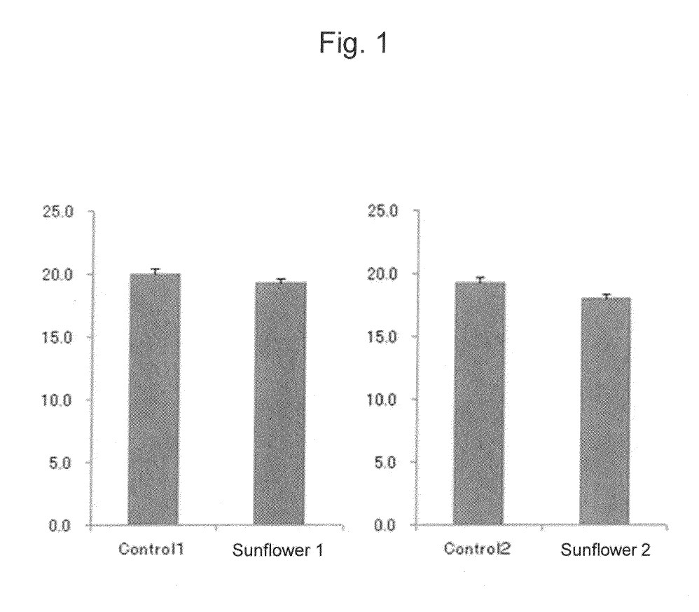

The results of the above test are shown as follows in FIG. 1.

Body Weight (g)

| | Control | Sunflower 1 (5% | Control | Sunflower 2 (10% |
|---|---|---|---|---|
| | 1 | incorporation) | 2 | incorporation) |
| a | 20.1 | | | |
| b | 18.9 | 18.3 | 18.1 | 17.2 |
| c | 20.9 | 19.8 | 19.6 | 18.0 |
| d | 20.2 | 19.3 | 20.1 | |
| e | | | 19.1 | 18.5 |
| f | | 19.5 | | 18.5 |
| Average | 20.0 | 19.2 | 19.2 | 18.1 |

The results did not demonstrate a significant difference. (The p-value is the probability of the occurrence of a mean difference between groups is represented by a percentage. When the p-value is 0.05, the accidental probability is 5%. When the probability is less than 5%, it signifies “the presence of a significant difference”). However, a body weight loss was observed for sunflower 2 (10% incorporation) to an greater extent than that for control 2 (cellulose powder), and the p-value of sunflower 2 was 0.066, which was close to a significant difference.

It is generally said that results cannot be easily obtained from body weight loss tests. Animals subjected to such a body weight loss test often lose weight and also become physically weak. However, the mice of the sunflower 2 group were completely healthy and exhibited no changes in appetite, urine output, motion, or the like.

Moreover, while the feces of mice fed with the crystallized cellulose-incorporated feed were large and yellow, the feces of mice fed with the sunflower stem internal fiber powder-incorporated feed were brown and black, and the sizes thereof were each about half those of mice fed with the crystallized cellulose-incorporated feed. The total amount of feces of mice fed with the sunflower-incorporated feed was about 10% higher than that of mice fed with the crystallized cellulose-incorporated feed. It was successfully observed that whereas crystallized cellulose has a discharge effect as a fiber, sunflower has not only the effect of oil discharge, but also has effects of water-soluble waste product discharge and fat-soluble waste product discharge and has a significant effect of rectal cleaning.

### Example 8

In a manner similar to that in Example 7, the white adipose tissues of mice divided into 4 groups were anatomized 3 weeks later and then white adipose tissues were analyzed.

White Adipose Tissue (g)

| | Control | Sunflower 1 (5% | Control | Sunflower 2 (10% |
|---|---|---|---|---|
| | 1 | incorporation) | 2 | incorporation) |
| a | 0.1954 | | 0.1434 | 0.1159 |
| b | 0.2213 | 0.1623 | 0.2370 | 0.1050 |
| c | | 0.1852 | 0.2257 | 0.1410 |
| d | 0.3734 | 0.2496 | 0.2107 | 0.1312 |
| e | | 0.2137 | | |
| f | 0.4206 | | | |
| Average | 0.3027 | 0.2027 | 0.2042 | 0.1233 |

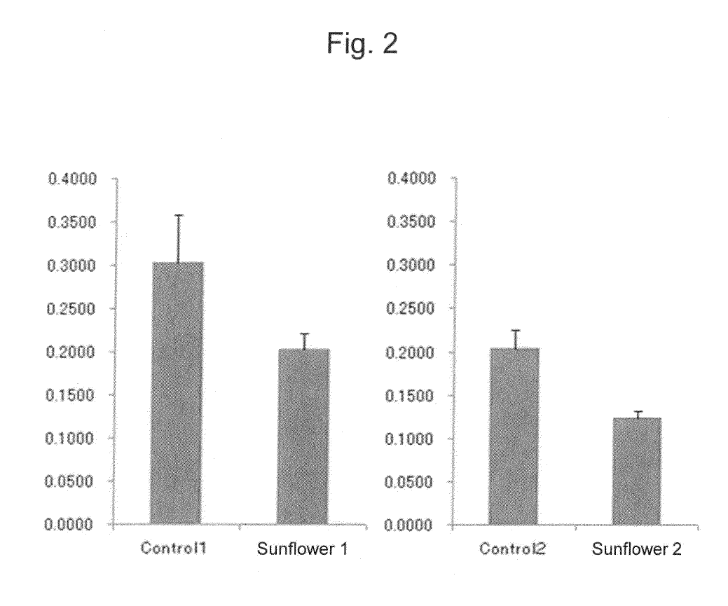

As is clear from the above numerical values and FIG. 2,

1) a difference between the control 1 group (fed with the feed containing the 5% crystallized cellulose powder) and the sunflower 1 group (fed with the feed containing the 5% sunflower stem internal fiber powder) did not reach a significant difference. A decrease was observed in white adipose tissue of the sunflower 1 group (p-value: 0.139).
2) A decrease in white adipose tissue was observed such that a significant difference (p-value: 0.01128) was observed between the control 2 group (fed with the feed containing the 10% crystallized cellulose powder) and the sunflower 2 group (fed with the feed containing the 10% sunflower stem internal fiber powder).
3) Compared with the control group of mice fed with the feed containing the crystallized cellulose, significantly higher effects (slimming effects) of reducing adipose tissues were obtained for the sunflower groups.

### Example 9

As in the results of Examples 7 and 8, slimming effects were exhibited in the case of 10% incorporation. Hence, the effect of decreasing the level of triglyceride (in blood) accounting for the majority of neutral fats was analyzed.

Triglyceride (mg/dl)

| | Control | Sunflower 2 |
|---|---|---|
| | 2 | (10% incorporation) |
| a | 110.1 | 84.4 |
| b | | |
| c | 117.4 | 71.6 |
| d | | |
| e | 100.9 | 77.1 |
| f | 100.9 | 80.7 |
| Average | 107.3 | 78.5 |

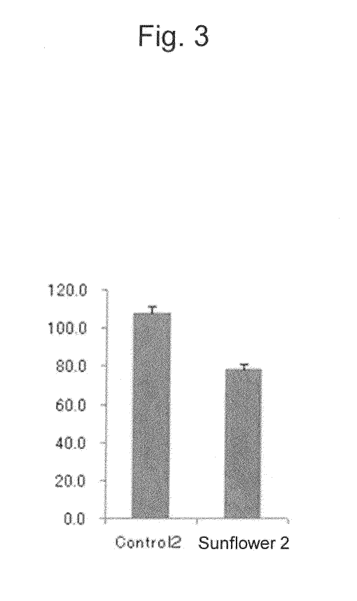

As shown in the above numerical values and FIG. 3, a significant difference (p-value: 0.0000993) indicating a large discharge effect was observed, compared with the control group of mice fed with the feed containing the crystallized cellulose.

### Example 10

Blood cholesterol level was measured in a test similar to those in Examples 7, 8, and 9.

Cholesterol (mg/dl)

| | Control | Sunflower 1 (5% | Control | Sunflower 2 (10% |
|---|---|---|---|---|
| | 1 | incorporation) | 2 | incorporation) |
| a | | 83 | 74.3 | 73.5 |
| b | | | 76.7 | |
| c | 94.9 | | 75.9 | 70.4 |
| d | 83.8 | 78.3 | | |
| e | 97.2 | 81.4 | 85.4 | 76.7 |
| f | | 81.4 | | 76.7 |
| Average | 92.0 | 81.0 | 78.1 | 74.3 |

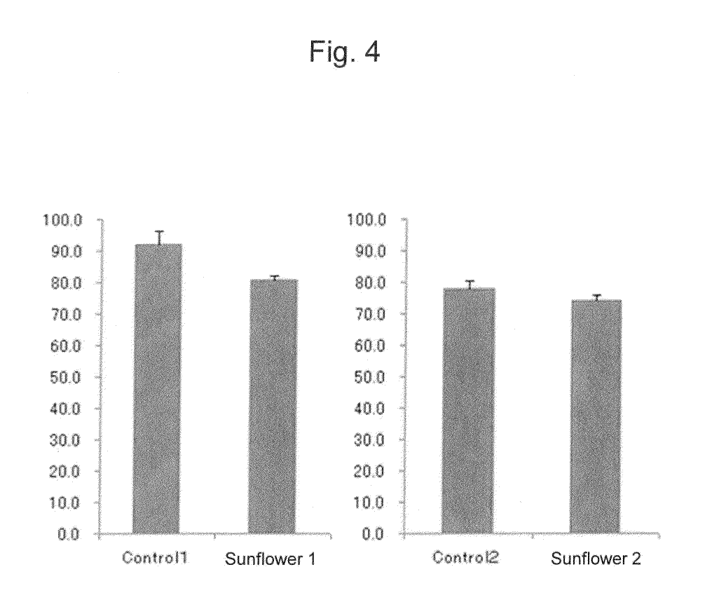

As is clear from the above numerical values and FIG. 4, a significant difference (p-value) of 0.030 was obtained between the control 1 group (fed with the feed containing the 5% crystallized cellulose) and the sunflower 1 group of mice fed with the feed containing the 5% sunflower.

### Example 11

The amounts of triglyceride and cholesterol in mouse feces were measured as in Example 7, and then discharge levels were measured. The daily mean feed intake was 2500 mg, and triglyceride (500 mg) accounted for 20% thereof.

TABLE 2

| | Amount of | |
|---|---|---|
| | triglyceride in | In vivo absorption of |
| | 1-day feces | triglyceride per day |
| 5% Cellulose powder | 18.7 mg | 96.3% |
| (crystallized cellulose) | | |
| incorporated | | |
| 10% Cellulose powder | 56.1 mg | 88.8% |
| (crystallized cellulose) | | |
| incorporated | | |
| 5% Sunflower pith | 59.3 mg | 88.1% |
| incorporated | | |
| 10% Sunflower pith | 87.7 mg | 82.5% |
| incorporated | | |

As is clear from Table 2, when the case of incorporation of the sunflower stem internal fibers (pith) was compared with the case of incorporation of cellulose powder, in vivo triglyceride absorption was lower in the case of incorporation of the sunflower stem internal fibers (pith). It was thus proven that the discharge of triglyceride accounting for the majority of neutral fats had increased.

TABLE 3

| | Cholesterol in | |
|---|---|---|
| | 1-day feces | |
| 5% Cellulose powder | 81.1 | mg |
| (crystallized cellulose) | | |
| incorporated | | |
| 10% Cellulose powder | 92.1 | mg |
| (crystallized cellulose) | | |
| incorporated | | |
| 5% Sunflower pith | 93.6 | mg |
| incorporated | | |
| 10% Sunflower pith | 116.0 | mg |

incorporated

Furthermore, as is clear from Table 3, it was proven that when the case of incorporation of the sunflower stem internal fibers (pith) was compared with the case of incorporation of the cellulose powder (crystallized cellulose), cholesterol discharge (to the outside of the body) was increased in the case of incorporation of the sunflower stem internal fibers (pith) rather than in the case of incorporation of the cellulose powder.

All publications, patents, and patent applications cited herein are incorporated herein by reference in their entirety.

1. A cholesterol and/or neutral fat excretion accelerator, comprising a lipophilic internal stem fiber powder separated from a sunflower stem.

2. A food comprising the cholesterol and/or neutral fat excretion accelerator of claim 1.

3. A supplement comprising the cholesterol and/or neutral fat excretion accelerator of claim 1.

4. (canceled)

5. A method of accelerating cholesterol discharge in a subject in need thereof comprising administering the cholesterol and/or neutral fat excretion accelerator of claim 1 to a subject in need thereof.

6. A method of accelerating neutral fat discharge in a subject in need thereof comprising administering the cholesterol and/or neutral fat excretion accelerator of claim 1 to a subject in need thereof.
